(19) United States
(12) Patent Application Publication
(10) Pub. No.: US 2016/0256485 A1
(43) Pub. Date: Sep. 8, 2016

(54) TREATMENT OR PREVENTION OF PULMONARY CONDITIONS WITH CARBON MONOXIDE

(71) Applicant: PROTERRIS, INC., Boston, MA (US)

(72) Inventors: Jeffrey Wager, Boston, MA (US); Joseph Wager, Boston, MA (US); Alex Stenzler, Garden Grove, CA (US)

(21) Appl. No.: 15/035,796

(22) PCT Filed: Nov. 14, 2014
(86) PCT No.: PCT/US14/65822

§ 371 Date: May 11, 2016

Related U.S. Application Data
(60) Provisional application No. 61/904,047, filed on Nov. 14, 2013.
(60) Provisional application No. 61/993,137, filed on May 14, 2014.

(51) Int. Cl. A61K 33/00; C12Q 1/37

(52) U.S. Cl. CPC A61K 33/00; C12Q 1/37; G01N 2333/96486

(57) ABSTRACT

A method of treating a patient having a lung condition that includes identifying a target haemoglobin-carbon monoxide level in the blood of the patient. The method also includes administering, to the patient, carbon monoxide at a first concentration for an initial time period, and measuring the haemoglobin-carbon monoxide level in the blood of the patient. The method also includes calculating, based on the measured haemoglobin-carbon monoxide level and the target haemoglobin-carbon monoxide level, a dose of carbon monoxide required to attain the target haemoglobin-carbon monoxide level within a determined time period. The method also includes administering, to the patient, the calculated dose of carbon monoxide for the determined time period to attain the target haemoglobin-carbon monoxide level in the blood of the patient.

### CROSS-REFERENCE TO RELATED APPLICATIONS

This application claims priority to U.S. Provisional Application No. 61/904,047 filed Nov. 14, 2013, titled “TREATMENT OR PREVENTION OF PULMONARY CONDITIONS WITH CARBON MONOXIDE”, and to U.S. Provisional Application No. 61/993,137 filed May 14, 2014, titled “TREATMENT OR PREVENTION OF PULMONARY CONDITIONS WITH CARBON MONOXIDE”, the entire disclosures of which are incorporated by reference herein in their entirety.

### BACKGROUND

While carbon monoxide (CO) gas has exhibited properties that make it an intriguing therapeutic candidate, CO gas inhalation strategies must avoid the potential toxicities of CO, especially if used in the treatment of lung conditions where patients may already have significant respiratory dysfunction. Ryter et al., Heme Oxygenase-1/Carbon Monoxide: From Basic Science to Theraneutic Applications Physiol Rev 86:583-650 2006.

While CO exposure has been shown to decrease airway neutrophil count and lung injury at early timepoints in models of acute lung injury, the effect was not sustained beyond the first 6 hours (Morse and Choi, Inhaled CO in the treatment of acute lung injury, Am J Physiol Lung Cell Mol. Physiol 294:L642-L643 (2008)), raising questions as to how or whether CO inhalation can be effective for chronic or even acute treatments of lung dysfunction.

Further, the prospects of CO therapy are hampered by an incomplete understanding of CO toxicology, even at low-doses. For example, toxic effects of chronic administration of CO may include extrapulmonary effects such as cardiac- and neurotoxicity. Mitchell et al., Evaluation of inhaled carbon monoxide as an anti-inflammatory therapy in a nonhuman primate model of lung inflammation. Am J Physiol Lung Cell Mol Physiol 299: L891-L897 (2010). This is in addition to the known clinical manifestations of CO poisoning, which include dizziness, drowsiness, vomiting, headache, and loss of motor coordination. Prolonged exposures to CO are known to cause respiratory difficulty, disorientation, chest pain, loss of consciousness, or coma and can ultimately result in death. Chronic exposure to low doses of CO may result in memory loss and other cognitive and neurological complications. Inhalation studies in rats show that CO can cause oxidative damage in the brain. Ryter et al., Heme Oxygenase-1/Carbon Monoxide: From Basic Science to Therapeutic Applications Physiol Rev 86:583-650 2006. Further, patients with underlying cardiovascular disease can be at significant risk upon CO poisoning, and such risks include myocardial ischemia or infarction.

Thus, treatment of acute or chronic lung conditions with CO gas, must administer a CO regimen to the patient to realize the therapeutic benefits while avoiding potential toxicity. Further, systems and methods are needed to safely and effectively administer CO regimens to patients, including patients that exhibit pre-existing pulmonary or cardiovascular conditions.

### DESCRIPTION OF INVENTION

In various aspects and embodiments, the invention provides for treatment of patients having acute or chronic inflammatory, hyperproliferative, or fibrotic conditions of the lungs. In some embodiments, the condition(s) include, but are not limited to, conditions such as pulmonary fibrosis (including Idiopathic Pulmonary Fibrosis (IPF)), asthma, emphysema, Chronic Obstructive Pulmonary Disease (COPD), pulmonary arterial hypertension (PAH), cystic fibrosis (CF), Acute Respiratory Distress Syndrome (ARDS), bronchiectasis, Ventilator-Assisted Pneumonia (VA), lung transplantation. In some embodiments, the patient has a condition defined by pulmonary fibrosis, such as IPF. In some embodiments, the lung condition permits the patient to breathe unassisted. In other embodiments, the patient may require metabolic support; for example assisted breathing, such as provided by a ventilator. In some embodiments, such as transplantation, the isolated donor organ may require metabolic support. Such support may be provided by normothermic or hypothermic extracorporeal perfusion utilizing a variety of solutions and gases. In some aspects, the CO may be delivered to the patient or organ as a gas. In other aspects, the CO may be dissolved in a fluid, which is delivered to the patient or organ.

In accordance with aspects of the invention, the patient having the lung condition undergoes treatment with inhaled CO, which in some embodiments is chronic CO treatment. The treatment regimen in various embodiments can be personalized based on one or more markers of fibrosis, such as MMP concentrations in patient samples (e.g., MMP-7) as well as target CO blood levels or blood CO-Hb levels.

In various embodiments, the patient has a forced vital capacity (FVC) of less than 80%, less than 70%, less than 60%, less than 55%, less than 50%, or less than 40%. In some embodiments, the patient has demonstrated a FVC decline of from 5% to 10% over a one week, one month, or six month period, and the CO-inhalation regimen is effective to slow or prevent further disease progression.

In some embodiments, the patient has MMP1, MMP7, and/or MMP8 blood levels (e.g., peripheral blood, serum, or plasma, etc.) that are substantially elevated compared to healthy controls. For example, in some embodiments, the patient has IPF, and baseline MMP7 levels are above about 12 ng/ml, or above about 10 ng/ml, or above about 8 ng/ml, or above about 5 ng/ml, or above about 3 ng/ml. In accordance with various embodiments, MMP7 levels are tested periodically as a measure of improvement, and are maintained at below about 8 ng/ml, and preferably below about 5 ng/ml or below about 3 ng/ml. For example, MMP7 levels may be substantially maintained at about control or subclinical levels.

In various embodiments, Carboxy-Hemoglobin (CO-Hb) is used as a marker to guide the CO administration regimen and/or the CO dosing protocol. For example, CO-Hb may be tested before, during, or after CO administration, using a blood test, percutaneous or transcutaneous device, or other device such as a pulse oximeter. CO-Hb in various embodiments can be used a marker for the end-point of a CO dose, and/or used to establish a CO-dosing protocol for a patient. In various embodiments, during CO administration CO-Hb is maintained below about 20%, below about 15%, below about 12%, below about 10%, or below about 8%. In some embodiments, each CO administration targets a CO-Hb endpoint, which may be below about 15%, below about 12%, below about 10%, or below about 8%, or may be about 7%, about 8%, about 9%, about 10%, about 11%, or about 12%. For example, the CO-Hb endpoint may be between about 8% and about 12%. In some embodiments, CO-Hb is maintained at the target level for a period of time during administration by adjusting the CO dose to achieve a steady state CO level. The steady state mode may be maintained for about 30 minutes, about 45 minutes, about 1 hour, about 2 hours, or about 3 hours. In some embodiments, for example in the case of hospitalized patients, CO-Hb is maintained at level of from about 5% to about 15% chronically with intermittent administration of CO, or about 8-12% in some embodiments. For example, the frequency of administration may be set to maintain a base CO-Hb level over time. This level may be substantially maintained for at least about one week, at least about two weeks, at least about one month, at least about two month, at least about six months, or for as long as the treatment is desired.

In some embodiments, the CO administration protocol comprises at least two concentration levels of CO gas; a relatively high level of CO to quickly reach a target CO blood level or CO-Hb level, and a maintenance level of CO to maintain the CO or CO-Hb endpoint for a period of time to provide the desired therapeutic effect. This latter concentration is referred to herein as the “steady state mode”. In such embodiments, the administration is safe and controlled to avoid toxic and/or undesired CO exposure, while reducing the time of the administration procedure considerably.

In various embodiments, the administration process comprises delivering CO gas at a constant alveolar concentration to a patient via inhalation. The delivery of CO gas to the patient reaches a steady-state during treatment, where equilibrium between the alveolar concentration and the patient's CO-Hb level is achieved. The steady-state uptake enables control of the delivered CO dose, and allows for safe administration of CO gas. In some embodiments, the steady-state mode (e.g., for maintaining a CO-Hb level of from 6% to about 12%) is continued for from 15 minutes to about 3 hours, or about 30 minutes, about 1 hour, about 1.5 hours, or about 2 hours.

There are many factors that can affect the uptake of carbon monoxide by a patient via inhalation. For example, some factors are related to characteristics associated with the patient, including but not limited to: changes in alveolar-capillary membrane (i.e. membrane factor); the pulmonary capillary blood volume; hemoglobin concentration; and total blood volume. Other factors associated with the patient can include CO back-pressure from endogenous CO production, and prior patient exposure to CO. The influence of these patient-related factors can vary based on the relative health of the patient. There are also non-patient factors that can affect the rate and extent of uptake by the patient, namely factors that can be controlled or at least influenced by the nature of the CO delivery system. For example, the most important of these factors is the alveolar concentration of CO. The alveolar concentration is the concentration of CO present in the gas in a patient's lungs during treatment. The alveolar CO concentration is a function of the movement of gases in the lung and also the partial pressure of CO in the gases in the lung. While the patient-related factors of CO uptake can be difficult to measure and account for, the alveolar concentration of CO can be held relatively constant through the use of the system and methods described herein. Therefore, by controlling the alveolar concentration of CO, fluctuations in the rate of CO uptake can be minimized or avoided.

The uptake of CO in humans is mostly dependent upon the concentration of the inhaled gas and the diffusing capacity of the lungs. The formation of HbCO on the basis of CO exposure has been described in a physiologically-based single-order pharmacokinetics model, and is referred to in the literature as the Cobum-Foster-Kane equation (i.e. the CFK equation or CFKE).

CFK Equation:

$$\frac{A [ HbCO ] t - BVco - PIco}{A [ HbCO ] O - BVco - PIco} = exp ( - \frac{tA}{VbB} )$$

Where:

A=PC_{O2}/M[HBO₂]

B=1/DL_{CO}+P_{L}/V_{A}

M=ratio of the affinity of blood for CO to that for O₂

[HbO₂]=ml of O₂ per ml of blood

[HbCO]₀=ml of CO per ml of blood at time t

[HbCO]₀=ml of CO per ml of blood at the beginning of the exposure interval

PC_{O2}=average partial pressure of oxygen in the lung capillaries in mmHg

V_{CO}=rate of endogenous CO production in ml/min

DL_{CO}=diffusivity of the lung for CO in ml/min×mmHg

P_{L}=barometric pressure minus the vapor pressure of water at body temperature in mmHg

Vb=pulmonary blood volume

PI_{CO}=partial pressure in the inhaled air in mmHg

V_{A}=alveolar ventilation in ml/min

t=exposure time in min

exp=2.7182, the base of natural logarithms raised to the power of the bracketed expression

According to the CFK equation, the time to reach an equilibration point between the alveolar concentration of CO and the body's stores can be relatively long, on the order of many hours in a healthy human (see, for example, FIG. 2 of Peterson J E, et al., Predicting the carboxyhemoglobin levels resulting from carbon monoxide exposures, J Applied Physiol Vol. 39(4):633-638 (1975). In a patient with diseased lungs, the time to reach a steady-state condition, that is, where the blood Hb-CO level reaches a plateau, can take even longer. However, if the HbCO level gets too high the patient can experience severe adverse effects or even death. Further, the concentration of CO in inhaled air can greatly affect the time needed to reach the desired steady-state concentration. For example, with a CO alveolar concentration of 25 ppm, it can take about 20 hours to reach an equilibration point, while at 1000 ppm, the time to reach steady-state can be shortened to between 2 and 3 hours. However, predicting equilibration points based on the CFK equation may be difficult, especially when Hb-CO measurements lie on the steeper region of a curve and all of the physiologic variables are unknown or cannot easily be measured.

In various embodiments, relatively high CO concentrations (e.g., at least 500 ppm, or at least 600 ppm, or at least 800 ppm, or at least 1000 ppm, or at least 1500 ppm, or at least 2000 ppm) are administered to the lungs in the initial period of the procedure in order to quickly achieve the desired CO-Hb level in the patient. Because all of the unknown physiologic influencers of the formation of CO-Hb are unknown values during this portion of the procedure, CO-Hb levels in the patient may be continuously or intermittently monitored to ensure that the patient's CO-Hb level does not exceed a safe level. Since there may be a fine line between safe and harmful levels of carboxyhemoglobin, it can be important in some embodiments to appropriately time CO-Hb testing, and to accurately predict CO-Hb endpoints to avoid CO toxicity.

The physiologically-based pharmacokinetics model associated with the CFK equation is limited in that it does not account for the existence of multiple physiologic compartments in the body, that is, it does not account for physiologic compartments other than the lungs. Benignus explored the arterial versus venous response to inhaled carbon monoxide [Benignus et al., 1994, J Appl Physiol. 76(4): 1739-45]. According to Benignus, not all subjects responded alike, and while the majority of subjects followed the CFK equation, some subjects substantially deviated from the CFK model. Benignus determined that antecubital venous Hb-CO levels were over-predicted and arterial Hb-CO levels were under-predicted, indicating the presence of at least one additional physiologic compartment. Further, Bruce et al. [2003, J Appl Phys. 95(3):1235-47] modeled CO-Hb responses to inhaled CO, and identified five compartments that can be considered in a model: lungs (alveolar), arterial blood, mixed venous blood, muscle tissue, and other soft tissues.

Therefore, in some embodiments, the CO administration protocol as described further comprises modeling inhaled CO uptake by considering at least one additional compartment other than the lungs, such as one or more of: muscle tissue, other soft tissue, arterial blood, and/or venous blood. In some embodiments the protocol comprises calculating a CO dose using the percentage of muscle mass in a subject as a variable. Conventional methods for CO administration use only body weight as a factor for dosage determination, which may result in missing other relevant factors. For example, by using only body weight, differences in the level of muscle mass between men and women may not be considered when specifying a CO dose amount for therapeutic treatment, even though differences in muscle mass can result in significant differences in CO uptake and/or storage.

Further still, systems based on pulsed dosing may miss additional relevant factors. In systems based on pulsed dosing, a volume of CO that is a fraction of the total dose per minute is set by the device operator and injected into the breathing circuit. The volume fraction injected is typically determined by the patient respiratory rate, so that equal portions of the specified dose are delivered with each breath. The dose per breath is typically fixed, independent of the size of the tidal volume, which is the volume of air displaced between normal inspiration and expiration. Therefore, when such a fixed CO volume is injected into a varying inspired volume of air, the concentration of CO in the alveoli will vary inversely to the size of the breath. Accordingly, at larger tidal volumes, the alveolar concentration of CO will fall and the uptake of CO will also fall. In general, patients do not breathe at a fixed tidal volume for every breath. There is a natural variation on a breath-by-breath basis, and, in addition, any variation in activity by a patient, e.g. at night when the patient is asleep, can change the alveolar concentration of CO in a pulsed-dose system. These variations can lead to significant variation in tidal volumes, which can result in a significant change in CO-Hb levels in the patient during treatment.

Accordingly, in a pulse-based dosing system and method of CO administration, the alveolar concentration is not constant. The alveolar concentration of one constituent in a mixture of gases is a function of the partial pressure of the constituent gas, i.e. the proportion of the constituent gas to all of the other gases in the mixture multiplied by the barometric pressure (i.e., less water vapor). For example, consider a system with pulsed addition of CO gas in which a patient inhaled a 25 mL bolus of a gas mixture containing 0.3% CO (3000 ppm) that was added to 700 mL of breath of air. If the patient had a functional residual capacity (FRC), i.e. the volume of air in the lungs at the end of a normal breath, of 1 liter, then when the inspired gas was mixed in the alveoli, the inspired gas would have been diluted to about 1.4 percent of the bolus concentration (25/(25+700+1000), or 43 ppm (a partial pressure of 3 mmHg at sea level). However, if the FRC was 400 mL instead of 1000 mL, the CO concentration would rise to almost 1.8%, or 53 ppm (3.7 mmHg). This change represents a 23 percent increase in CO concentration that could result in a 2 percent increase in the blood HbCO level, which could be enough to produce adverse health effects. Accordingly, the greater the delivered dose in a pulse-based system, the greater the potential variability. In acute disease states and in particular in patients on mechanical ventilators, even larger changes in FRC could be present, and such changes could result in even more significant swings in partial pressure that would affect uptake and might compromise patient safety. Alternatively, when the inspired gas is precisely premixed and delivered as a constant concentration, independent of respiratory rate, FRC, or tidal volume, there is little or no fluctuation in alveolar gas concentration.

In certain aspects, the CO administration protocol provides a constant alveolar concentration of carbon monoxide. In these aspects, the protocol safely delivers a specified concentration of CO, for example in ppm levels, to either mechanically-ventilated or spontaneously breathing patients having a chronic or acute pulmonary condition. The use of constant alveolar concentration dosing assures that the patient's alveolar concentration will remain the same, and that the patient's Hb-CO level will reach a steady state during the treatment and enables relatively easy adjustments for better control of the delivered dose. Thus, in some embodiments, the concentration of CO is adjusted during treatment to maintain a constant alveolar concentration.

In related aspects, the invention further provides methods for predicting CO-Hb level during CO administration. Considering that inter-patient differences, for example diffusing capacity, cardiac output, endogenous carbon monoxide production and pulmonary capillary blood volume, can result in significant differences in carboxyhemoglobin levels for the same dosage level of CO, a method to accurately predict the carboxyhemoglobin level at any point in time of exposure would be of great value. In some embodiments, the administration process (or the personalization of the CO regimen) comprises a reverse calculation of DL_{CO} (the Diffusing capacity or Transfer factor of the lung for carbon monoxide), to more accurately predict the desired CO dose. For example, a first concentration of CO is administered for a period of time, such as from 5 minutes to about 30 minutes, such as from about 10 minutes to about 25 minutes (or for about 10 minutes, about 15 minutes, about 20 minutes, or about 30 minutes in various embodiments). At the end of that period CO-Hb is measured. From the actual measurement, DL_{CO} can be calculated from the CFK equation by substitution and solving for DL_{CO} because all of the other variables are known, and DL_{CO} accounts for the balance of the difference from predicted value. With the physiologically derived DL_{CO} (including the miscellaneous physiologic factors), one can accurately predict the CO-Hb using the CFK equation at any point in time at the same inspired CO, or make a change in inspired CO and predict the CO-Hb at any other point in time. In some embodiments, a tested CO-Hb level is input into the delivery system by a user or an associated CO-Hb measuring device, and the time and further CO dose to reach the desired end-point is automatically adjusted by the delivery system.

For example, in some embodiments a first concentration of CO is administered to the patient for from 5 minutes to about 30 minutes (as described above), and CO-Hb is measured either using an associated device or by drawing blood for testing. From the CO-Hb measurement, the dose of inhaled CO to reach the desired CO-Hb endpoint at a particular time is determined. This determination can comprise calculating DL_{CO}. This subsequent CO dose over the determined period of time is provided to the patient to reach the CO-Hb end-point, which in some embodiments is maintained as a steady-state level for continued CO administration.

In some embodiments, each administration of CO is a predetermined regimen to reach a selected CO-Hb endpoint (e.g., steady-state concentration), and maintain that end-point for a period of time. This regimen may be empirically tested for the patient, and determined based on a set of criteria, and then subsequently programmed into the delivery system. In these embodiments, cumbersome and invasive blood tests are avoided, which further renders the treatment suitable for home care. Further, in some embodiments, the method or system does not rely on continuous CO or CO-Hb measurements, but relies on a specified regimen personalized for the patient. Thus, in some embodiments, CO dosing scheme (e.g., ppm over time, including steady-state administration steps) is determined in a personalized manner in a clinical setting with CO-Hb testing (e.g., blood test), and the selected dosing schedule used going forward (either in the clinic or outpatient setting) without CO-Hb monitoring. In some embodiments, the CO-Hb is tested after administration at least once per year, or once every 6 months, or every other month, or once a month, to ensure that the dosing regimen remains appropriate for the patient, based on, for example, improving or declining health (e.g., lung function). In some embodiments, a less invasive pulse oximeter can be used to monitor CO levels when using a personalized regimen as described herein.

In various aspects, the invention uses systems to reliably control the CO administration process. For example, the methods may employ a CO dosing system to regulate the quantity of carbon monoxide which is delivered from a carbon monoxide source to the delivering unit. In various embodiments, the system comprises a sensor that determines the concentration of carbon monoxide in the blood of the patient, including spectroscopic or other methods, and/or means to measure carbon monoxide in the gas mixture expired from a patient (e.g., by spectroscopic methods or gas chromatography). The system may further comprise a control unit for comparing the actual CO blood concentration with a preset desired value, and subsequently causing the dosing unit to regulate the amount of carbon monoxide delivered to the patient to obtain a concentration in the patient's blood corresponding to the preset desired value. The control unit may perform a program control, a sensor control, or a combined program/sensor control.

CO-Hb levels can be determined by any method. Such measurements can be performed in a non-invasive manner, e.g., by spectroscopic methods, e.g., as disclosed in U.S. Pat. Nos. 5,810,723 and 6,084,661, and the disclosure of each is hereby incorporated by reference. Invasive methods which include the step of taking a blood sample, are employed in some embodiments. An oxymetric measurement can be performed in some embodiments, e.g., as disclosed in U.S. Pat. No. 5,413,100, the disclosure of which is hereby incorporated by reference.

Although the best-known reaction of carbon monoxide incorporated in a human or animal body is the formation of carboxyhemoglobin, it can also interact with other biological targets such as enzymes, e.g. cytochrome oxidase or NADPh. Activity measurements regarding these enzymes may thus also be employed for calculating the carbon monoxide concentration in the blood, and used as end-points for CO administration as described herein.

There is an equilibrium regarding the distribution of carbon monoxide between blood and the respired gas mixture. Another method for determining the blood concentration of CO is the measurement of the carbon monoxide concentration in the expired air of a patient. This measurement may be done by spectroscopic methods, e.g., by ultra red absorption spectroscopy (URAS), or by gas chromatography. This method of determination is well-established in medical art for the determination of the diffusing capacity of the lungs of a patient.

In some embodiments, the CO administration procedure comprises: setting a target Hb-CO level in the blood of the patient to be treated; administering CO gas at a first concentration while measuring the HbCO level in the patient's blood; reducing the CO level to a second concentration while continuing to monitor the patient's HbCO level; and continuing the administration of CO gas at the second concentration for a desired a period of time, referred to herein as steady-state mode. For example, CO gas may be delivered via inhalation for a relatively brief initial period, for example 30 minutes to 1 hour, at an inhaled CO concentration of 100 to 600 ppm until a desired blood level of CO is reached, for example about 7%, about 8%, about 9%, or about 10% HbCO, or other target concentration described herein. The time to reach the targeted value can vary significantly according to the patient's lung function or other factors, and methods for predicting the time required to reach the target blood level can be inaccurate or inconsistent. In some embodiments, the CO gas is delivered at an initial CO concentration until the desired HbCO level is achieved, instead of setting a specific time period for the CO delivery at the first CO concentration. In another embodiment, the concentration of CO gas delivered to the patient during the initial period may be more than 600 ppm, or less than 100 ppm.

In one embodiment, the concentration of the CO being administered can be adjusted during administration based on real-time feedback from a pulse oximeter, or any other type of sensor that can directly or indirectly measure CO levels in a patient's blood. In such an embodiment, a target level of HbCO is set instead of setting a target level for the CO concentration being administered. The CO concentration can be automatically adjusted by the control system, depending on how the patient's HbCO level are responding to the CO concentration being delivered. For example, if the patient's HbCO level is increasing faster than expected, in comparison to pre-set reference parameters, the control system can lower the CO concentration being administered. In one embodiment, the control system uses the CFK equation to calculate the DL_{CO} and then calculates the change in inspired CO concentrations.

In one embodiment, once the desired HbCO level in the patient's blood is achieved, CO gas is delivered to the patient at a second, lower concentration for a desired period of treatment time (e.g., from about 30 minutes to about 3 hours). This period of delivery at the second CO concentration is generally referred to herein as the steady-state delivery mode. In such embodiments, the CO concentration is reduced to the level needed to maintain the target HbCO level at steady-state without exposing the patient to toxic levels of CO.

In some embodiments, the system and method may further comprise other features, such as an alarm or warning system, an automatic shutoff feature, or an automated transition to a steady-state delivery mode. In one embodiment, when the HbCO level in the patient or the CO concentration in the breathing circuit is greater than the desired target level, the system of the present invention can institute an alarm or warning message to alert the operator, patient, or other person, of the deviation of the measured variable from a set point or target level. The alarm can be in the form of any visual, audio, or tactile feedback that would be suitable for informing a person of the deviation. In another embodiment, the system and method comprises an automatic shutoff feature that stops delivery of CO gas to the patient when the HbCO level or CO concentration in the breathing circuit exceeds a specified level.

In yet another embodiment, the system or method of the present invention comprises an automated transition to a steady-state delivery mode, wherein the concentration of CO gas being delivered to the patient is automatically reduced to a lower concentration once the desired level of HbCO in the patient has been achieved.

In various embodiments, the CO gas is administered to the patient at from 20 to 500 ppm CO during the steady state mode. For example, the CO gas may be from 20-200 ppm of CO, or 50 to 150 ppm CO, 50 to 100 ppm CO in some embodiments. In some embodiments, CO gas during the steady state mode is less than 100 ppm. In other embodiments, the CO gas may be from 100 to 400 ppm, such as from 100 to 300 ppm or 100 to 200 ppm. In some embodiments, the CO gas is more than 200 ppm CO.

In various embodiments, the system or method involves a control system suitable for the delivery of a constant CO alveolar concentration to a patient. The system can deliver the desired CO concentration independent of any change in breathing pattern, flow rate, respiratory rate, or tidal volume in a subject. In one embodiment, the gas delivery control unit is connected to at least one gas source, e.g. a mixture of CO in air, oxygen, or an inert gas such as nitrogen, and can control the delivery of the gas source to the breathing circuit of a subject. In one embodiment, comprises a high speed (e.g. 1 ms) dynamic mixing subsystem that tracks the flow of breathing gases going to the patient, and injects carbon monoxide from a high concentration source tank, for example a gas source with a concentration of 1000-10,000 ppm CO, or 3000 to 5000 ppm CO in some embodiments, directly into the breathing circuit in the proportion needed to maintain the desired concentration.

In one embodiment, the system also comprises a pulse oximeter sensor that measures the HbCO level in a patient's blood. By non-limiting example, the pulse oximeter may be a Massimo RAD57 pulse oximeter. In another embodiment, the system comprises a sensor that measures the concentration of CO gas in the patient breathing circuit. In yet another embodiment, the system of the present invention comprises any type of sensor, other than a pulse oximeter, that is suitable for measuring or determining the HbCO level in a subject's blood. By non-limiting example, the sensor may be an Instrumentation Laboratories IL-182 CO-Oximeter.

In one embodiment, the system or method involves at least one central processing unit (CPU) or microprocessor for use in monitoring or controlling the CO gas concentration in the breathing circuit, the HbCO level in the patient, or any other variable necessary for operation of the system and methods described herein. In cases where it is desired to maintain a target HbCO level in a patient after the target level is reached, the device can automatically decrease the inspired CO gas concentration to the level required to maintain the desired steady-state HbCO concentration. The system may also comprise alarm or warning systems that can trigger warning messages or an automated shut-off, as described herein. In one embodiment, the measured HbCO values are continuously read by a CPU, and if the HbCO level rises above the pre-set threshold, the CPU can sound an alarm, display a warning message on the control unit, and/or send a signal to turn off delivery of CO gas to the breathing circuit.

In one embodiment, the system has at least two CPUs, wherein one CPU is used for monitoring the mixing of gases, for example air and CO, and the flow of CO-containing gas to the patient. In such an embodiment, a second CPU monitors other variables, for example the concentration of CO or oxygen in the inspired gas, the HbCO level measured by the pulse oximeter, or any other variable associated with the system. Further, the system may monitor the pressure in one or more gas source tanks feeding gas to the control system of the present invention, in order to assure that continuous therapy, i.e. gas flow, is provided.

In some embodiments, MMP7 levels are tested at least once weekly or once monthly, and the patient's treatment adjusted to substantially maintain MMP7 levels near subclinical levels (e.g., less than about 6 ng/ml or less than about 5 ng/ml or less than about 4 ng/ml), and CO-Hb tested in connection with CO administration to substantially maintain a target CO-Hb level of from 5 to 15%, and around 10 to 14% during or immediately after CO administration.

In some embodiments, the patient is undergoing therapy with one or more pharmaceutical interventions (e.g., for IPF), which provides additional and/or synergistic benefits with the CO regimen.

For example, in some embodiments, the patient receives nitric oxide treatment, in addition to CO. In some embodiments, the patient is undergoing therapy with one or more of the following: one or more anti-inflammatory and/or immunomodulating agents, an anticoagulant, endothelin receptor antagonist, vasodilator, antifibrotic, cytokine inhibitor, and kinase inhibitor.

In various embodiments, the patient is undergoing therapy with a corticosteroid, such as prednisone or prednisolone. In some embodiments, the patient is undergoing treatment with azathioprine and/or N-acetyl-cysteine (NAC). In some embodiments, the patient is undergoing double or triple therapy with a corticosteroid (e.g., prednisone), azathioprine, and/or NAC. In still other embodiments, the patient is undergoing treatment with an antifibrotic, such as pirfenidone or Interferon-γ, or TNF-α inhibitor (e.g., etanercept). In these or other embodiments, the patient is undergoing treatment with one or more anticoagulants, such as warfarin or heparin. In these or other embodiments, the patient is undergoing treatment with one or more tyrosine kinase inhibitors, such as BIBF 1120 or Imatinib. In these or other embodiments, the patient is undergoing treatment with one or more phosphodiesterase inhibitors, such as sildenafil, or endothelin receptor antagonist, such as bosentan, ambrisentan, or macitentan. Other therapies that may provide synergistic or additive results with CO therapy include inhibitors of IL-13, CCL2, CTGF, TGF-β1, αvβb integrin, LOXL (e.g., neutralizing monoclonal antibody against IL-13, CCL2, CTGF, TGF-β1, αvβb integrin, LOXL).

In other example, including where the patient has asthma, COPD, or IPF, the patient is undergoing therapy with a bronchodilator, leukotriene inhibitor, glucocorticosteroid, mucolytic, or oxygen treatment. For example, the patient may be undergoing treatment with a short acting or long acting beta agonist, anticholinergic, or an oral or inhaled steroid. In some embodiments, the patient is undergoing therapy with albuterol, theophylline, budesonide, formoterol, fluticasone/salmeterol (e.g., Advair), or montelukast (e.g., Singulair).

In some embodiments, including embodiments in which the patient has cystic fibrosis, the patient undergoes treatment with one or more of an antibiotic, mucolytic, or bronchodilator.

Carbon monoxide compositions in various embodiments comprise 0% to about 79% by weight nitrogen, about 21% to about 100% by weight oxygen and about 1000 to about 10.000 ppm CO carbon monoxide. More preferably, the amount of nitrogen in the gaseous composition comprises about 79% by weight, the amount of oxygen comprises about 21% by weight and the amount of carbon monoxide comprises about 4000 to 6000 ppm.

A gaseous CO composition may be used to create an atmosphere that comprises CO gas. The gases can be released into an apparatus that culminates in a breathing mask or breathing tube, thereby creating an atmosphere comprising CO gas in the breathing mask or breathing tube, ensuring the patient is the only person in the room exposed to significant levels of CO.

CO levels in an atmosphere can be measured or monitored using any method known in the art. Such methods include electrochemical detection, gas chromatography, radioisotope counting, infrared absorption, colorimetry, and electrochemical methods based on selective membranes (see, e.g., Sunderman et al., Clin. Chem. 28:2026 2032, 1982; Ingi et al., Neuron 16:835 842, 1996). Sub-parts per million CO levels can be detected by, e.g., gas chromatography and radioisotope counting. Further, CO levels in the sub-ppm range can be measured in biological tissue by a midinfrared gas sensor (see, e.g., Morimoto et al., Am. J. Physiol. Heart. Circ. Physiol 280:H482 H488, 2001). CO sensors and gas detection devices are widely available from many commercial sources.

In delivering CO to patients or in other applications at concentrations ranging from about 0.001 to about 3,000 ppm, gaseous compositions may be prepared by mixing commercially available compressed air containing CO (generally about 1% CO) with compressed air or gas containing a higher percentage of oxygen (including pure oxygen), and then mixing the gasses in a ratio which will produce a gas containing a desired amount of CO therein. Alternatively, compositions may be purchased pre-prepared from commercial gas companies. In some embodiments, patients are exposed to oxygen (O₂ at varying doses) and CO at a flow rate of about 12 liters/minute in a 3.70 cubic foot glass exposure chamber. To make a gaseous composition containing a pre-determined amount of CO, CO at a concentration of 1% (10,000 ppm) in compressed air is mixed with >98% O₂ in a stainless steel mixing cylinder, concentrations delivered to the exposure chamber or tubing will be controlled. Because the flow rate is primarily determined by the flow rate of the O₂ gas, only the CO flow is changed to generate the different concentrations delivered to the exposure chamber or tubing. A carbon monoxide analyzer (available from Interscan Corporation, Chatsworth, Calif.) is used to measure CO levels continuously in the chamber or tubing. Gas samples are taken by the analyzer through a portion the top of the exposure chamber of tubing at a rate of 1 liter/minute and analyzed by electrochemical detection with a sensitivity of about 1 ppb to 600 ppm. CO levels in the chamber or tubing are recorded at hourly intervals and there are no changes in chamber CO concentration once the chamber or tubing has equilibrated.

In some embodiments, the CO-containing gas is supplied in a high pressure vessel containing between about 1000 and about 10,000 ppm of CO, and in some embodiments at about 3.000 to about 7000 ppm of CO, or about 4,000 to 6,000 ppm CO, or about 5000 ppm of CO, and connected to a delivery system. The delivery system measures the flow rate of the air that the patient is breathing and can inject a proportionally constant flow rate of the CO-containing gas into the breathing gas stream of the patient so as to deliver the desired concentration of CO in the range of 0.005% to 0.05% to the patient to maintain a constant inhaled CO concentration.

In another embodiment, the flow of oxygen-containing air that is delivered to the patient is set at a constant flow rate and the flow rate of the CO-containing gas is also supplied at a constant flow rate in proportion to the oxygen-containing air to deliver the desired constant inhaled CO concentration.

The pressurized gas including CO can be provided such that all gases of the desired final composition (e.g., CO, He, Xe, NO, CO₂, O₂, N₂) are in the same vessel, except that NO and O₂ cannot be stored together. In some embodiments, the gas composition contains at least one noble gas. Optionally, the methods of the present invention can be performed using multiple vessels containing individual gases. For example, a single vessel can be provided that contains carbon monoxide, with or without other gases, the contents of which can be optionally mixed with the contents of other vessels, e.g., vessels containing oxygen, nitrogen, carbon dioxide, compressed air, or any other suitable gas or mixtures thereof.

A CO-containing gas mixture may be prepared as above to allow passive inhalation by the patient using a facemask or tent. For example, the delivery system may provide a steady stream of gas composition for inhalation. The concentration inhaled can be changed and can be washed out by simply switching over to 100% O₂. Monitoring of CO levels would occur at or near the mask or tent with a fail-safe mechanism that would prevent too high of a concentration of CO from being inhaled.

In some embodiments, the CO gas is administered to the patient by a ventilator. In some embodiments, the CO gas is administered to the patient or donor organ via an extracorporeal perfusion machine. For example, during the ischemic phase of lung transplant surgery. In some embodiments, the patient is able to spontaneously breathe, and the CO gas is administered without any ventilation assistance.

The CO gas may be delivered from about 1 to about 7 times weekly, including once, twice, or three times weekly. In some embodiments, the CO treatment is delivered about once, about twice, or about three times monthly. In some embodiments, the CO treatment may be administered for at least 6 months, or at least 1 year, or at least 2 years, or at least 5 years, or more, or as long as the benefits of CO treatment as disclosed herein are exhibited. In each such embodiment, CO may be administered from 1 to 3 times on each day of treatment. In some embodiments, the dosing regimen is as disclosed in U.S. Pat. No. 8,778,413 titled “DOSING REGIMENS AND METHODS OF TREATMENT USING CARBON DIOXIDE”, the disclosure of which is incorporated herein by reference in its entirety.

In some embodiments, the CO treatment is carried out as disclosed in U.S. Pat. No. 8,128,963 titled “METHODS FOR TREATING ISCHEMIC DISORDERS USING CARBON MONOXIDE”, the disclosure of which is incorporated herein by reference in its entirety.

In some embodiments, oxygen gas (e.g., without CO) is delivered between CO treatments, or as needed. For example, oxygen gas may be delivered to the patient from 1 to 7 times per week, between CO treatments, and for about 10 minutes to about 1 hour per oxygen treatment.

In some embodiments, the patients receiving the chronic CO therapy are monitored for lung function and disease progression, and the CO regimen adjusted as needed.

In some embodiments, serum MMP7 levels are monitored, with a rise in MMP7 levels suggesting that higher or more frequent doses of CO are required.

In some embodiments, the patient's lung capacity is monitored using a spirometer or similar device. In these embodiments, the patient can self-monitor lung function, and adjust the frequency or length of CO administration accordingly.

In some embodiments, serum oxyHb and/or carboxyHb are measured yearly or monthly, to monitor or manage possible long term toxicity of CO. Carbon monoxide binds to hemoglobin preferentially compared to oxygen. Even at low levels COHb can lead to oxygen deprivation of the body causing tiredness, dizziness and unconsciousness.

COHb has a half-life in the blood of 4 to 6 hours, but in cases of poisoning, this can be reduced to 70 to 35 minutes with administration of pure oxygen. In addition, treatment in a Hyperbaric Chamber for CO poisoning can be used. This treatment involves pressurizing the chamber with pure oxygen at an absolute pressure close to three atmospheres allowing the body's fluids to absorb oxygen and to pass free oxygen on to hypoxic tissues instead of the crippled hemoglobin bonded to CO.

### EXAMPLES

### Example 1

### Study of Back Calculation of DLCO and Predicting COHb at 60 Minutes

To test the ability to predict CO-Hb levels at 60 minutes based on blood measurements of COHb at earlier time points, an experiment was performed in a S. pneumoniae model induced in four juvenile baboons. Using measured COHb levels after 10, 20, 30, 40, and 50 minutes of 200 ppm CO administration, a computer program generated in MATLAB (Mathworks) was used to back calculate the estimated DL_{CO} (including unmeasured physiologic variables) using the CFK equation (Coburn et al. JCI, 43: 1098-1103, 1964: Peterson et al. JAP, Vol. 39, No. 4, 633-638, 1975). Then using the estimated DL_{CO} and measured time point CO-Hb levels, the computer then used the CFK equation to predict the CO-Hb level after a 60 min. CO exposure. There was good correlation between the predicted and measured COHb levels (Table below). It was determined that this method can be used to predict the 60 min CO-Hb level with high accuracy (R2=0.9878) using the 20 min COHb level.

| Time Point | 60 Min predicted COHb | | |
|---|---|---|---|
| (Min) | minus actual COHb | SD | 95% CI |
| 10 | −0.777% | 1.07 | (−3.4, 1.8) |
| 20 | 0.28% | 0.43 | (−0.4, 0.97) |
| 30 | −0.05% | 0.18 | (−0.33, 0.23) |
| 40 | −0.13% | 0.06 | (−0.23, −0.03) |
| 50 | −0.11% | 0.05 | (−0.2, −0.02) |

### Example 2

### Study of Inhaled Carbon Monoxide to Treat Idiopathic Pulmonary Fibrosis

The primary outcome measure is the change in MMP7 serum level over 3 months of treatment. Serum MMP7 concentrations in peripheral blood are easily measurable and reflect changes in the alveolar microenvironment. Thus, we have chosen to study mean serum MMP7 concentrations after three months of CO treatment as a surrogate biomarker of the effect of inhaled CO administration on disease progression.

A secondary outcome measure is Total Lung Capacity (TLC). Total lung capacity (TLC) is a major clinical determinant of restrictive lung disease in practice, with TLC measurement below the 5th percentile of the predicted value indicative of a restrictive ventilatory defect.

Another secondary outcome measure is diffusing capacity for carbon monoxide. Interstitial changes associated with IPF can worsen diffusing capabilities across the alveolar-capillary membrane. As a result, diffusing capacity of carbon monoxide is an important outcome to assess architectural distortion and resultant decrements in diffusing capabilities.

Another secondary outcome measure is six minute walk distance. The six minute walk distance is commonly used both in research studies and in clinical practice as a measure of functional capabilities and changes in six minute walk distance and oxygen use during testing over time often reflect clinically relevant disease progression. The distance traveled during six minutes (meters) will be measured in accordance with published guidelines.

St. George's Respiratory Questionnaire (SGRQ) will be used, which is a validated self-reported instrument. In this instrument, scores range from 0 to 100, with higher scores reflective of worse quality of life.

The primary intervention will be inhaled CO at 100-200 ppm administered two times weekly for two hours per dose to complete 12 weeks of treatment.

The placebo comparator will be Oxygen 21% (room air oxygen concentration).

Idiopathic pulmonary fibrosis (IPF) is an interstitial lung disease characterized by destruction of normal epithelial structure, proliferation of fibroblasts, and deposition of connective-tissue matrix proteins. There are currently no effective therapies for IPF. Preclinical studies of inhaled low dose carbon monoxide (CO) have shown that this biologically active diatomic gas possesses properties that make it a viable novel therapy for IPF. CO therapy has been well tolerated in Phase I and Phase II human trials to date. This phase II study is designed to investigate whether IPF patients show evidence of decreased peripheral blood levels of MMP7 and stability of secondary indicators of disease progression after 3 months of inhaled therapy.

Inclusion Criteria:
Adults above the age of 18 and equal to or below the age of 85
Diagnosis of IPF by biopsy or ATS/ERS/ALAT Guidelines (Am J Respir Crit Care Med Vol 183, pp 788-824, 2011)
FVC greater than or equal to 50% predicted, greater than or equal to one month off all medications prescribed for IPF

Exclusion Criteria:
Evidence of active infection within the last month
Significant obstructive respiratory defect
Supplemental oxygen required to maintain an oxygen saturation over 88% at rest
History of myocardial infarction within the last year, heart failure within the last 3 years or cardiac arrhythmia requiring drug therapy
History of smoking within 4 weeks of screening
Pregnancy or lactation
Participation in another therapeutic clinical trial

What is claimed is:

1. A method of treating a patient having a lung condition, comprising:
identifying a target haemoglobin-carbon monoxide level in the blood of the patient;
administering, to the patient, carbon monoxide at a first concentration for an initial time period;
measuring the haemoglobin-carbon monoxide level in the blood of the patient;
calculating, based on the measured haemoglobin-carbon monoxide level and the target haemoglobin-carbon monoxide level, a dose of carbon monoxide required to attain the target haemoglobin-carbon monoxide level within a determined time period; and
administering, to the patient, the calculated dose of carbon monoxide for the determined time period to attain the target haemoglobin-carbon monoxide level in the blood of the patient.

2. The method of claim 1, further comprising administering, to the patient, carbon monoxide at a second concentration for a treatment time period, wherein the target haemoglobin-carbon monoxide level achieved in the blood of the patient is substantially maintained during the treatment time period.

3. The method of claim 1, wherein the lung condition is selected from pulmonary fibrosis, asthma, emphysema, Chronic Obstructive Pulmonary Disease (COPD), pulmonary arterial hypertension (PAH), cystic fibrosis (CF), Acute Respiratory Distress Syndrome (ARDS), bronchiectasis, Ventilator-Assisted Pneumonia (VA), and lung transplantation.

4. The method of claim 3, wherein the lung condition is pulmonary fibrosis.

5. The method of claim 4, wherein the pulmonary fibrosis is Idiopathic Pulmonary Fibrosis (IPF).

6. The method of claim 1, wherein the initial time period is between about 5 minutes and about 1 hour.

7. The method of claim 1, wherein the target haemoglobin-carbon monoxide level is between about 7% and about 15%.

8. The method of claim 1, wherein the target haemoglobin-carbon monoxide level is between about 8% and about 12%.

9. The method of claim 1, wherein the first concentration is between about 100 ppm and about 2000 ppm.

10. The method of claim 1, wherein the treatment time period is between about 30 minutes and about 3 hours.

11. The method of claim 1, wherein the second concentration is between about 20 ppm and about 500 ppm.

12. The method of claim 1, wherein the administering during the initial time period, or the administering during the determined time period, or both, is carried out using a ventilator.

13. The method of claim 1, wherein the administering during the initial time period, or the administering during the determined time period, or both, is carried out using an extracorporeal perfusion machine.

14. The method of claim 1, wherein the administering during the initial time period, or the administering during the determined time period, or both, is carried out without assisted breathing.

15. The method of claim 1, wherein the patient has a forced vital capacity (FVC) of less than 80%.

16. The method of claim 1, wherein the patient has a forced vital capacity (FVC) of less than 40%.

17. The method of claim 1, wherein the patient has elevated levels of at least one of matrix metalloproteinase-1 (MMP1), matrix metalloproteinase-7 (MMP7), or matrix metalloproteinase-8 (MMP8) blood levels.

18. The method of claim 1, the calculating the dose of carbon monoxide including calculating the diffusing capacity of the lung of the patient for carbon monoxide.
